(19) United States
(12) Patent Application Publication
(10) Pub. No.: US 2005/0074873 A1
(43) Pub. Date: Apr. 7, 2005

(54) Tissue culture vessel

(76) Inventors: Michael S. Shanler, Somerville, MA (US); Kevin M. Hughes, Burlington, MA (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(21) Appl. No.: 10/922,679

(22) Filed: Aug. 19, 2004

Related U.S. Application Data
(60) Provisional application No. 60/501,495, filed on Sep. 9, 2003.

(51) Int. Cl. C12M 1/24

(52) U.S. Cl. 435/304.3; 435/288.1

(57) ABSTRACT

A tissue culture vessel includes opposed top and bottom walls and sidewalls extending between the top and bottom walls. The top wall is formed with a septum aperture and a resealable septum is mounted in the septum aperture to permit access to interior regions of the tissue culture vessel by a medical device for accessing tissue grown in the vessel. The top wall may also include a membrane aperture and a membrane may be mounted in the membrane aperture for providing gas communication to interior portions of the tissue culture vessel.

### RELATED APPLICATIONS

This application claims the benefit of U.S. Provisional Application No. 60/501,495 filed on Sep. 9, 2003 which is hereby incorporated by reference in its entirety.

### BACKGROUND OF THE INVENTION

1. Field of the Invention

The invention relates generally to tissue culture vessels. More particularly, the invention relates to vessels for growing cells, microorganisms and tissue in a culture medium and then conveniently accessing materials in the vessel.

2. Description of the Related Art

Tissue culture vessels are used widely in laboratories for many purposes. For example, tissue culture vessels are used to culture microorganisms or tissues in a culture medium or agar. The microorganisms or tissues are permitted to grow under controlled conditions. The tissues then may be accessed periodically and tested.

May tissue culture vessels are of generally prismatic shape with a plurality of upstanding sidewalls extending between opposed top and bottom walls. The sidewalls generally are constructed so that the length and width of the vessel exceed the height. As a result, the bottom wall of the vessel defines a fairly large surface area relative to the volume of the vessel. A tubular neck typically is formed at one of the sidewalls of the vessel to provide access to the interior. The outer surface of the neck may be formed with an array of threads for threadedly receiving a cap.

Tissue culture vessels typically are employed by removing the cap from the neck of the vessel and depositing a selected amount of a liquid growth medium in the vessel. Cells or tissue then are inserted into the vessel through the opening in the neck and the cap is replaced on the neck. Several such vessels typically are arranged in a fairly dense array and at a controlled location in a laboratory. The vessels may be accessed periodically to assess the growth of the cells or tissue in the vessel. The access to the interior of the vessel may be achieved by removing the cap from the neck of the vessel and inserting a scrapper, swab or pipette through the neck sufficiently for accessing the tissue in the growth media. This procedure is effective but very inefficient and not well suited to automated laboratory equipment.

U.S. Pat. No. 4,334,028 shows a tissue culture vessel with a frangible zone formed in the top wall of the vessel. The frangible zone is defined by a region of reduced thickness that may be cut or broken to access the interior of the vessel. An area of the top wall near the frangible zone defines a hinge. Thus, the frangible zone effectively defines a trap door that can be rotated about the hinge to access the interior of the vessel.

Some tissues must be exposed to air to grow properly. U.S. Pat. No. 5,047,347 shows a vessel with a gas permeable membrane incorporated into a wall of the vessel or a portion of the closure. A cover is hingedly mounted near the gas permeable membrane for selectively covering the membrane. The hinged cover for the membrane shown in U.S. Pat. No. 5,047,347 is not well suited for use with automated laboratory testing equipment.

Many laboratory analyses can be completed with smaller amounts of cells or tissues and smaller volumes of growth medium. Thus, more laboratory tests can be completed within a smaller area of a laboratory. However, it is necessary to locate the smaller volume of liquid growth media and the smaller areas of cells or tissues in a predictable manner within a vessel so that the cells or tissues can be harvested easily.

Laboratory equipment is available for collecting small amounts of liquid with a robotic device. For example, multi-well plate assemblies are employed in laboratories and have an array of small wells arranged in a rectangular matrix. A typical multi-well plate may include 96 wells arranged in an 8×12 rectangular matrix. Laboratory equipment also includes robotic pipette devices for automatically entering access ports of the multi-well plate assembly for removing small amounts of liquid in the respective wells. The robotic device then moves the array of pipettes to another location so that the small amount of liquid collected on the respective pipettes can be analyzed. The above-described tissue culture vessels are not well suited for use with robotic devices, and hence are used primarily with less efficient manual procedures for growing and harvesting tissue cultures.

### SUMMARY OF THE INVENTION

The invention relates to a tissue culture vessel with a top wall, a bottom wall and a plurality of sidewalls extending between the top and bottom walls. A hollow neck extends from one of the sidewalls and provides communication with the interior of the vessel. Exterior portions of the neck may include cap attachment structures, such as an array of threads. Thus, a cap may be attached removably to the neck for closing the interior of the vessel. One of the walls of the vessel spaced from the neck is formed with at least one aperture and a self-sealing septum extends across the aperture. The self-sealing septum may be formed from an elastomeric material and may have a slit extending at least partly through the elastomeric material. Alternatively, the septum may be formed with a cross-cut defining a generally X-shaped pair of cuts each of which extends at least partly through the septum. The septum could be configured to be accessed by a pipette tip or other pointed implement for accessing liquid and cells in the vessel.

The vessel may further include a second aperture covered by a membrane that permits a flow of gas across the membrane without permitting liquid to flow across the membrane. The membrane preferably is disposed on a surface of the vessel that will remain dry, and hence permits gas exchange, oxygenation or humidity control in the vessel.

The top and bottom walls of the vessel may define major surface areas as compared to areas defined by any of the sidewalls. Additionally the bottom wall may include footprint alignment features for positioning the vessel in a specified location and orientation to permit access by a robotic device. For example, the bottom surface may have structural features for fixing and orienting the vessel relative to alignment tiles on a robot deck. The robot then can be programmed to access the vessel at the self-sealing septum for automatically testing or harvesting the tissue or cells being grown in the vessel.

The bottom wall of the vessel can be configured to define a trough in which liquid growth medium will collect due to forces of gravity. The trough defined in the bottom wall of the vessel may be registered with the self-sealing septum.

The self-sealing septum may be configured to permit a single pipette or other collection device to pass through the septum and into an area of the vessel at which liquid media will collect. Alternatively, the self-sealing septum may permit a plurality of pipettes or other collection devices to pass simultaneously through the self-sealing septum. For example, the self-sealing septum may be elongated and may have an elongate slit or a linear array of cross-cuts. The trough defined by the bottom wall of the vessel may extend substantially along an axis defined by the elongate self-sealing septum.

The self-sealing septum and/or the membrane may be incorporated into one or more caps mounted in the apertures in the wall of the vessel. The self-sealing septum and the membrane can be mounted in the same cap.

In still other embodiments, the vessel can be configured to be stored on one of the small side surfaces. Thus, the major surfaces define sides aligned substantially vertically. In these embodiments, the self-sealing septum and the membrane may be provided in a side surface substantially opposite the surface on which the vessel will be supported.

### BRIEF DESCRIPTION OF THE DRAWINGS

FIG. 1 is an exploded perspective view of a culture vessel in accordance with the invention.

FIG. 2 is a perspective view, partly in section, of the vessel of FIG. 1 in the fully assembled condition.

FIG. 3 is a top plan view of the vessel.

FIG. 4 is s cross-sectional view taken along line 4-4 in FIG. 3.

FIG. 5 is a top plan view similar to FIG. 3, but showing a cover with an alternate septum.

FIG. 6 is an exploded perspective view of an alternate vessel in accordance with the invention.

FIG. 7 is a top plan view of a vessel similar to the vessel shown in FIG. 6, but showing an alternate septum.

FIG. 8 is an exploded view of a cap and vessel according to a further embodiment.

FIG. 9 is a perspective view of an alternate cap.

FIG. 10 is an exploded perspective view of the tissue culture vessel in proximity to a robot deck.

### DETAILED DESCRIPTION

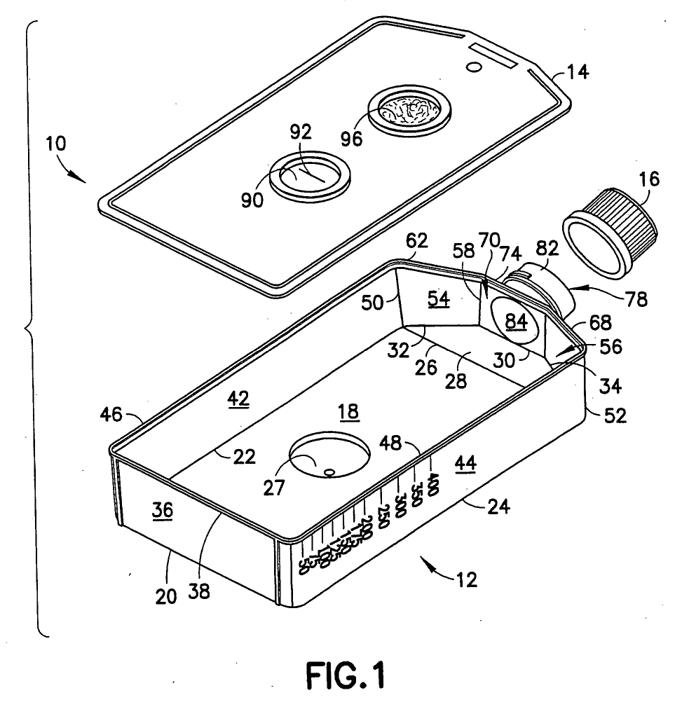

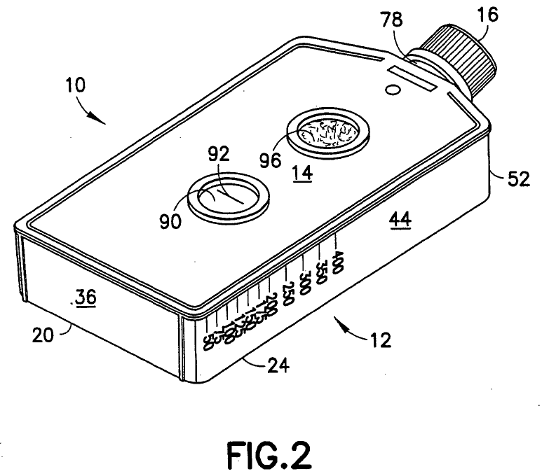

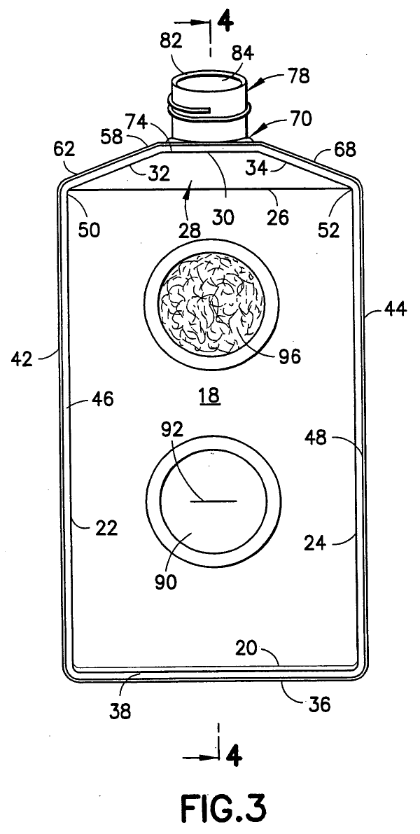

A tissue culture vessel in accordance with the invention is identified generally by the numeral 10 in FIGS. 1-4. Tissue culture vessel 10 is a generally hexagonal container with a base 12, a cover 14 and a cap 16. Base 12 is formed unitarily from a plastic material, and preferably a polystyrene. Base 12 includes a substantially planar rectangular bottom wall 18 with a back end 20, first and second sides 22 and 24 and a front end 26.

A substantially conically generated trough 27 extends down at a location on bottom wall 18 substantially centrally between back and front ends 20 and 26 and substantially centrally between sides 22 and 24. A substantially planar isosceles trapezoidal ramp 28 extends unitarily from front end 26 of bottom wall 18 and is aligned to bottom wall 18 at an obtuse angle of about 150°. Hence, the plane of trapezoidal ramp 28 defines an incline of about 30° when bottom wall 18 is supported on a horizontal surface. Front end 26 of bottom wall 18 defines the longer of two parallel bases for trapezoidal ramp 28. Ramp 28 further includes a shorter parallel base end 30 and first and second equal sides 32 and 34 that converge from end 26 toward end 30.

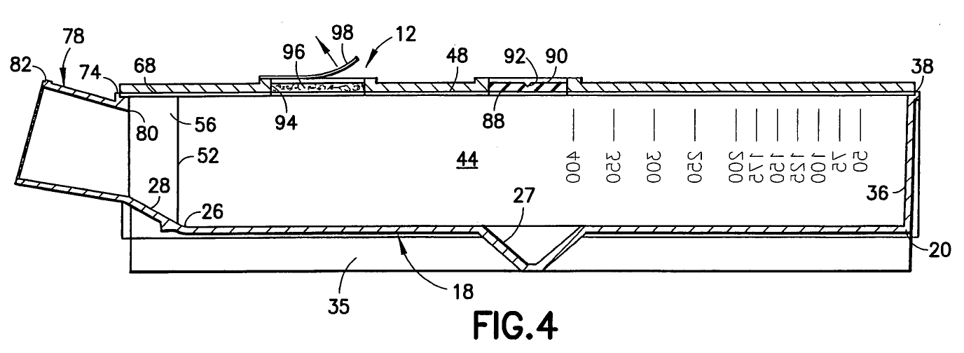

Bottom supports 35 extend down from bottom wall 18, as shown in FIG. 4. Bottom ends of the bottom supports 35 define a plane extending substantially parallel to planar portions of bottom wall 18. The plane defined by the bottom ends of the bottom supports 35 is coplanar with or lower than the bottom of bottom trough 27. Bottom supports 35 also define an outer periphery substantially in the shape of a rectangle.

Base 12 of vessel 10 includes a substantially rectangular back wall 36 that projects orthogonally from bottom wall 18 at a location adjacent back end 20 of bottom wall 18. Back wall 36 includes a top edge 38 aligned substantially parallel to bottom wall 18. Base 12 also includes first and second substantially parallel rectangular sidewalls 42 and 44 that extend orthogonally from bottom wall 18 at locations adjacent first and second sides 22 and 24 respectively. First and second sidewalls 42 and 44 include top edges 46 and 48 respectively that are parallel to bottom wall 18 and substantially coplanar with top edge 38 of back wall 36. Sidewalls 42 and 44 have front ends 50 and 52 substantially aligned with opposed sides of front end 26 of bottom wall 18.

Base 12 further includes first and second substantially planar transition walls 54 and 56 that converge toward one another from front ends 50 and 52 of first and second sidewalls 42 and 44 respectively. First transition wall 54 is substantially trapezoidal and has a top edge 62 that is substantially in the plane defined by top edges 38, 46 and 48. Second transition wall 56 also is substantially trapezoidal and includes a top edge 68 substantially in the plane defined by top edges 38, 46, 48 and 62.

Base 12 of vessel 10 further includes a substantially planar front wall 70 aligned substantially orthogonal to the plane defined by bottom wall 18. Front wall 70 is substantially rectangular and has first and second sides coincident with the front ends of first and second transition walls 54 and 56 respectively. Front wall 70 further includes a top edge 74 that extends between top edges 62 and 68 of first and second transition walls 54 and 56. Top edge 74 lies in the plane defined by top edges 38, 46, 48, 62 and 68.

Base 12 of vessel 10 further includes a generally tubular neck 78 that extends forwardly from front wall 70. Neck 78 includes an open rear end 80 at front wall 70 that communicates with the region of base 12 above bottom wall 18 and ramp 28. Neck 78 further includes a front end 82 and a tubular passage 84 extending between rear end 80 and front end 82. Portions of neck 78 adjacent front end 82 are substantially cylindrically generated and exterior regions of neck 78 adjacent front end 82 include an array of external threads for threaded engagement of cap 16.

Cover 14 of vessel 10 is substantially planar and defines a hexagon with a shape that permits cover 14 to rest on top edges 38, 46, 48, 62, 68, and 74 of base 12 or to nest slightly with the vertical walls of base 12. Cover 14 may be secured in position on base 12 by appropriate application of adhesive or by a known bonding technique, such as ultrasonic welding.

Cover 14 includes a septum aperture 88 at a location aligned with conically generated trough 35 in bottom wall 18, as shown in FIG. 4. A self-sealing septum 90 is secured in septum aperture 88. Septum 90 is formed from an elastomeric material and is provided with a longitudinal slit 92 aligned substantially along a diameter of aperture 88. Slit 92 may extend entirely through septum 90 or partly through septum 90 and will enable an access device, such as a pipette to be passed through septum 90 for collecting a tissue culture. However, the elastomeric material of septum 90 will reseal upon removal of the access device.

Cover 14 also is formed with a membrane aperture 94 and a membrane 96 is mounted securely in membrane aperture 94, as shown in FIG. 4. Membrane 96 is formed from a material that will permit gas exchange or oxygenation across the otherwise substantially impervious walls of vessel 10. Membrane 96 preferably is sealed initially by a removable sealing layer 98. Sealing layer 98 can be kept in place for those situations where gas exchange is not desired or can be removed at an appropriate time for situations where gas exchange is desired.

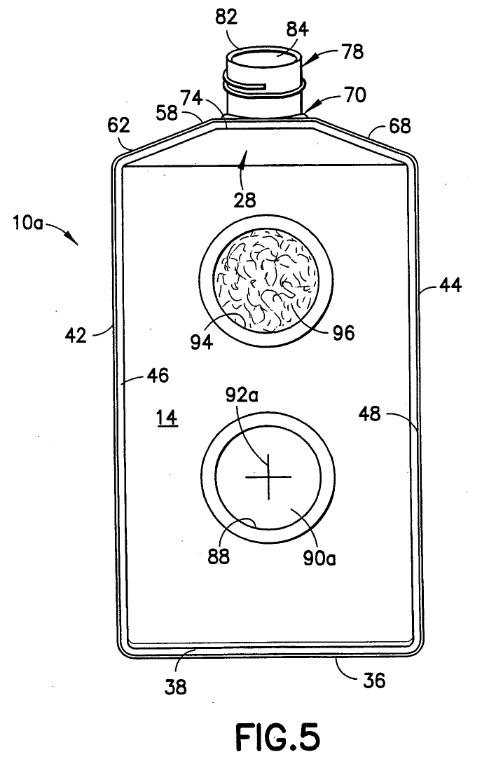

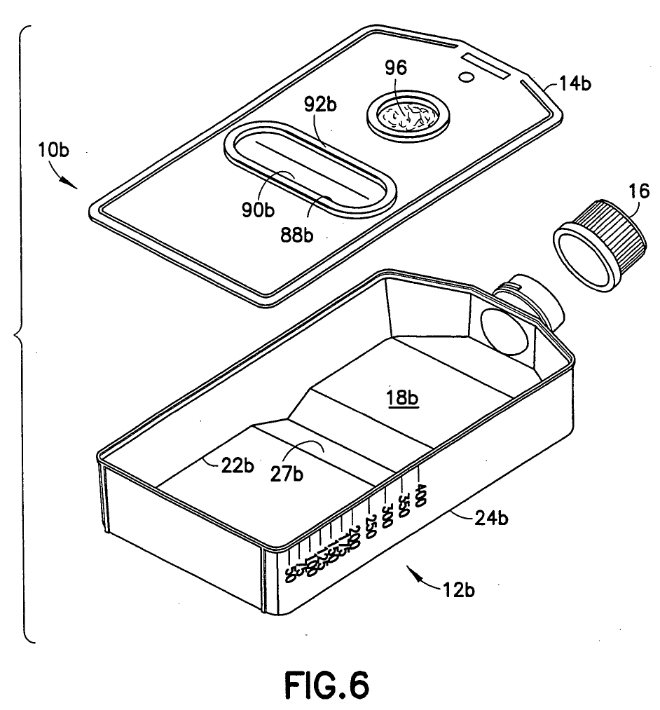

FIG. 5 shows a vessel 10a that is substantially identical to vessel 10 described and illustrated in FIGS. 1-5. In particular, vessel 10a includes a base 12 identical to the base 12 of vessel 10 and a cap 16 identical to the cap of vessel 10. Vessel 10a further includes a cover 14 that is substantially identical to the cover of vessel 10. However, septum aperture 88 of cover 14 is provided with a septum 90a with a cross-cut 92a as shown in FIG. 6. Cross-cut 92a may provide a more preferable access for certain types of access devices.

Another alternate culture vessel is identified generally by the numeral 10b in FIG. 6. Culture vessel 10b include a base 12b, a cover 14b and a cap 16. Base 12b is very similar to base 12 of culture vessel 10 described and illustrated above. However, bottom wall 18b is provided with an elongate trough 27b that extends substantially continuously between side edges 22b and 24b. All other aspects of base 12b are identical to base 12, and are not described again. Cover 14b is very similar to cover 14. However, cover 14b includes an elongate generally elliptoid septum aperture 88b and a correspondingly configured septum 90b. Septum 90b is provided with an elongate resealable slot 92b disposed and aligned to register substantially with elongate trough 27b in bottom wall 18b. Slot 92b enables a plurality of access devices, such as pipettes to be passed simultaneously through slot 92b for obtaining a plurality of tissue or cell cultures simultaneously. Slot 92b then will reseal simultaneously for access again at a later stage. All other aspects of culture vessel 10b are substantially identical to culture vessel 10.

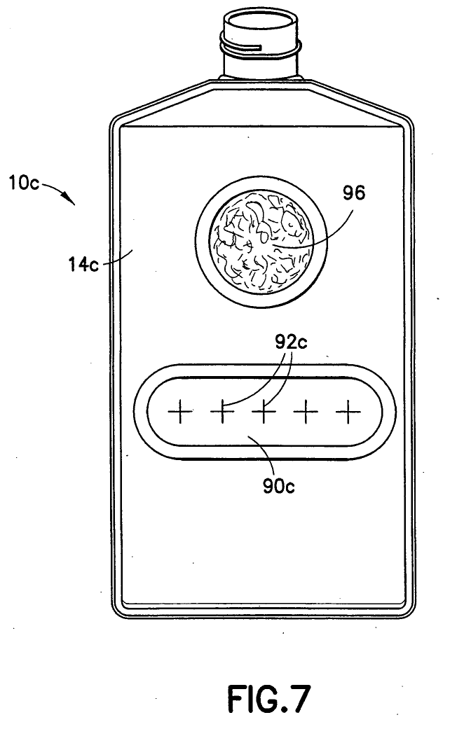

A variation of culture vessel 10b is identified by the numeral 10c in FIG. 7. Culture vessel 10c includes a base and a cap substantially identical to the culture vessel 10b. The culture vessel in FIG. 7 further includes a cover 14c substantially identical to the cover 14b described and illustrated with respect to FIG. 6. However, cover 14c includes a septum 90c with a plurality of spaced apart cross-cuts 92c. Each cross-cut 92c may be substantially identical to the cross-cuts 92a illustrated in FIG. 5. Cross-cuts 92c may facilitate access for certain types of access devices, while enabling simultaneous access by a plurality of such devices.

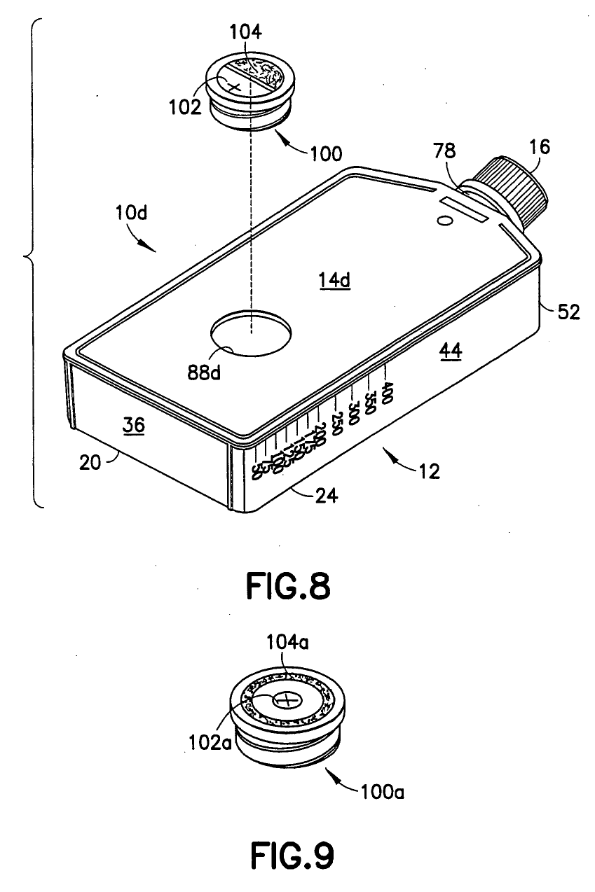

FIG. 8 shows a culture vessel 10d similar to culture vessel 10 described and illustrated above. In particular, culture vessel 10d includes a base 12 and a cap 16 substantially identical to the corresponding parts of the culture vessel 10 described and illustrated with respect to FIGS. 1-5. However, culture vessel 10d has a cover 14d with only one aperture 88d and a threaded cap 100 is mounted in aperture 88d. A septum 102 and a membrane 104 are mounted in cap 100. Septum 102 and membrane 104 each are substantially semi-circular. However other shapes can be provided, such as a circular septum 102a and an annular membrane 104a, as shown in FIG. 9. These designs enable the type of septum (e.g., straight cut or cross-cut) to be changed for a particular application. Similarly, the type of membrane can be changed for a particular application. Alternatively, a solid cap can be engaged in aperture 88d for those situations where no septum or no membrane is desired. Similarly, a cap with only a split septum or only a membrane can be employed.

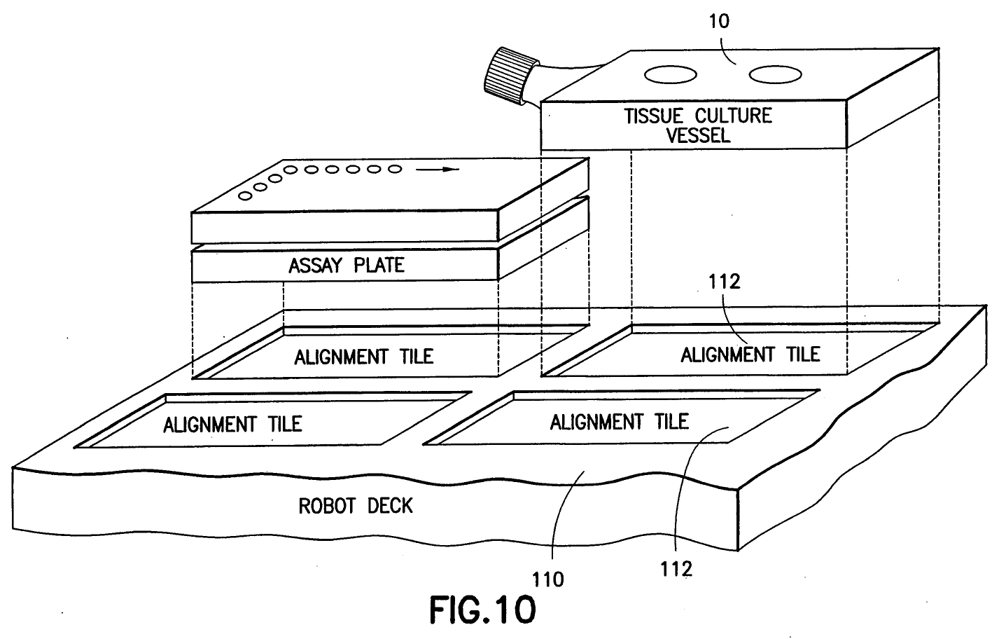

The culture vessel of the subject invention is well suited for use with automotive robotic devices for accessing the interior of the culture vessel and obtaining samples of cell or tissue cultures. For example, as shown in FIG. 10, culture vessel 10 can be used with a robot deck 110 that has a plurality of rectangular alignment tile recesses 112. Bottom supports 35 of culture vessel 10 are dimensioned to nest in alignment tile 112 to provide a specific arrangement of X,Y coordinates for culture vessel 10.

1. A tissue culture vessel having a base with a bottom wall and a plurality of sidewalls extending up from said bottom wall, a cover extending across said sidewalls and opposed to said bottom wall, said cover being formed with at least one opening extending therethrough and a septum extending across said opening for permitting access to interior portions of said vessel by a medical device.

2. The tissue culture vessel of claim 1, wherein the septum is formed from a material that is resealable after access by the medical device.

3. The tissue culture vessel of claim 2, wherein said septum is formed with at least one slit extending at least partly through said septum.

4. The tissue culture vessel of claim 3, wherein said at least one split comprises two slits intersecting one another for defining a cross-cut in said septum.

5. The tissue culture vessel of claim 1, wherein said aperture in said cover is substantially circular.

6. The tissue culture vessel of claim 5, wherein said bottom wall of said base is formed with a trough substantially opposed to said aperture in said cover for collecting liquid media at a location below said septum.

7. The tissue culture vessel of claim 1, wherein said aperture is an elongate aperture.

8. The tissue culture vessel of claim 7, wherein said bottom wall of said base includes an elongate trough formed therein and substantially aligned with said elongate aperture in said cover for collecting liquid media in said trough at locations substantially aligned with said septum.

9. The tissue culture vessel of claim 7, wherein said septum includes at least one slit extending at least partly through said septum for facilitating access by a medical device.

10. The tissue culture vessel of claim 9, wherein said at least one slit comprises a plurality of pairs of intersecting slits at spaced apart locations along said septum.

11. The tissue culture vessel of claim 1, wherein said septum is secured in a cap and wherein said cap is mounted to said aperture in said cover.

12. The tissue culture vessel of claim 11, wherein one said sidewall of said base is formed with a hollow neck for pouring liquid media into or out of said vessel.

13. The tissue culture vessel of claim 12, further comprising a closure releasably mounted to said neck.

14. The tissue culture vessel of claim 1, wherein said cover further includes a membrane for providing gas communication between the interior of said vessel and ambient surroundings.

15. The tissue culture vessel of claim 14, wherein said aperture is a septum aperture and wherein said cover further comprises a membrane aperture, said membrane being mounted to said membrane aperture.

16. The tissue culture vessel of claim 1, further comprising a cap mounted to said aperture in said cover, said septum being mounted in a portion of said cap, a second portion of said cap being formed with a membrane for providing gas communication to interior portions of said tissue culture vessel.

17. A tissue culture vessel having a bottom wall, a top wall opposed to said bottom wall and a plurality of sidewalls extending between said top and bottom walls, a hollow neck formed at one of said sidewalls and providing communication to interior portions of said vessel for pouring a liquid media into or out of said vessel, a closure securely mounted to said neck for selectively closing said neck, said top wall of said tissue culture vessel having a septum aperture formed therethrough, a septum mounted in said septum aperture and having at least one slit extending at least partly therethrough to permit communication of a medical device with interior portions of said vessel, a membrane aperture formed through said top wall and a breathable membrane being mounted in said membrane aperture.

18. The tissue culture vessel of claim 17, wherein said bottom wall of said vessel is formed to define a trough substantially aligned with said septum aperture.

19. The tissue culture vessel of claim 18, wherein said septum aperture and said trough are elongated, and wherein said at least one slit is configured for receiving a plurality of medical devices.
